(19) United States
(12) Patent Application Publication
(10) Pub. No.: US 2008/0206351 A1
(43) Pub. Date: Aug. 28, 2008

(54) Malodor Reduction of Cosmetic Products

(75) Inventors: Lin Yang, Woodbridge, CT (US); Georgia Shafer, Southbury, CT (US); Alexander Lips, New Canaan, CT (US)

Correspondence Address: UNILEVER PATENT GROUP, 800 SYLVAN AVENUE, AG West S. Wing, ENGLEWOOD CLIFFS, NJ, 07632-3100, US

(73) Assignee: CONOPCO, INC., D/B/A UNILEVER, Englewood Cliffs, NJ (US)

(21) Appl. No.: 11/678,290

(22) Filed: Feb. 23, 2007

(51) Int. Cl. A61K 8/02

(52) U.S. Cl. 424/502; 424/489; 424/76.1; 514/770

(57) ABSTRACT

Malodor free skin care compositions are described. The skin care compositions comprise an adsorbable solvent and an insoluble particle in an adsorbable solvent-insoluble particle complex. The complex is suitable to adsorb components with malodor to yield a skin care composition free of offensive odors like those generated from the oxidation of conjugated linoleic acid.

### FIELD OF THE INVENTION

The present invention is directed to a skin care composition and a method for reducing malodor in skin care compositions. More particularly, the invention is directed to skin care compositions comprising insoluble particle that is suitable to adsorb compounds that have been proven to yield offensively unpleasant odors in compositions, like lotions, creams and body washes. The insoluble particle is preferably used with the adsorbable solvent suitable to be adsorbed on to the insoluble particles (i.e., an adsorbable solvent) whereby compositions that contain insoluble particle and the adsorbable solvent surprisingly show a reduction in malodor, and especially, a reduction in malodor originating from compounds suitable to, for example, hydrogen bond to the insoluble particle and/or be scavenged by the adsorbable solvent which in is part of an adsorbable solvent-insoluble particle complex. Furthermore, the compositions of the present invention, which comprise an adsorbable solvent-insoluble particle complex, unexpectedly show a reduction in malodor that is greater than the sum of malodor reduction for compositions that only contain insoluble particle and only contain adsorbable solvent.

### BACKGROUND OF THE INVENTION

A wide variety of skin care compositions tend to generate malodors after coming into contact with air, bacteria, skin or combinations of the same for prolonged periods of time. In fact, many skin care compositions comprise actives that, for example, oxidize, thereby generating volatile components that result in malodor. Attempts at reducing malodor in skin care compositions have been made. For example, fragrances have been used in skin care compositions to mask malodors. Use of fragrances, however, is not always desirable since many consumers wish to use skin care compositions that are free of fragrances, due to various skin sensitivities and allergies. Also, fragrances within a product tend to have a shorter life than the product itself. Therefore, malodor masking may not be achieved during an entire product life.

There is increasing interest to develop skin care compositions that are free of malodor, and especially, skin care compositions that are free of malodor and that are suitable to yield the characteristic benefit they are known to produce. This invention, therefore, is directed to a skin care composition comprising insoluble particles and preferably an adsorbable solvent. The skin care compositions made according to this invention are surprisingly free of malodor originating from compounds that, for example, are suitable to hydrogen bond to the insoluble particle and/or are scavenged by the adsorbable solvent in an adsorbable solvent-insoluble particle complex.

### Additional Information

Efforts have been disclosed for making cosmetic compositions. In World Application No. WO 93/18130, malodor personal cleansing bars with zeolite are described.

Other efforts have been disclosed for making cosmetic compositions. In U.S. Application No. 2006/0135385 A1, toilet bar compositions with pyran odor masking agents are described.

Still other efforts have been disclosed for making consumer product compositions with reduced odor. In European Patent Application No. EP 0063899 A2, fabric conditioning compositions with aluminum chlorohydrate are described.

Even other efforts have been disclosed for making cosmetic compositions. In Japanese Application No. JP 2004290573 A, deodorants having elasticity and flexibility are described whereby the same uses clay as a swelling agent.

None of the additional information above describes a skin care composition that has insoluble particle and adsorbable solvent whereby the composition is free of malodor originating from compounds suitable to, for example, hydrogen bond to the insoluble product and adsorb to the adsorbable solvent in an adsorbable solvent-insoluble particle complex.

### SUMMARY OF THE INVENTION

In a first aspect, the present invention is directed to a method for reducing malodor in a skin care composition comprising the steps of:
(a) formulating the skin care composition with a component with a malodor or an ingredient that can degrade to yield a component with a malodor; and
(b) including in the skin care composition an insoluble particle and an adsorbable solvent, the component with a malodor being one suitable to hydrogen bond with the insoluble particle and be scavenged by the adsorbable solvent in an adsorbable solvent-insoluble particle complex
   wherein the insoluble particle has a surface area from about 75 to about 3500 m²/g and the adsorbable solvent has a solubility parameter distance from the component with the malodor, Ra, of less than about 20.

In a second aspect, the present invention is directed to a malodor-free skin care composition made according to the method described in the first aspect of this invention.

Additional aspects of the present invention will more readily become apparent from the description and examples which follow.

Skin, as used herein, is meant to include all skin on the face and body. Skin care composition is meant to mean a composition that may be applied to skin and/or hair as a leave on and/or rinse off composition. Such a skin care composition is not limited with respect to the form it takes, and therefore, can be, for example, a bar, liquid, gel, stick, roll-on formulation, cream, aerosol or non-aerosol spray, fabric (e.g., non-woven textile)-applied formulation, mousse, lotion, ointment, cosmetic, cosmetic remover, foundation, conditioner or shampoo. The skin care composition is not limited in use and can, for example, lighten, moisturize, clean, nourish, or reduce wrinkles or oil on skin as well as clean, condition or be used to sculpt hair.

Ingredient that can degrade to yield a component with malodor, as used herein, is meant to mean any ingredient that is often used in a topical composition like those that provide a benefit to hair or skin when, for example, being topically applied. Component with a malodor is meant to include, for example, heterocompounds like low molecular weight (<C₁₀) aldehydes and amines that can be found in skin care compositions. Free of malodor or malodor-free is meant to mean free of odor that is offensive, and for example, free of an odor generally produced by aldehydes such as hexanal. Component with malodor and malodor component are meant to be the same. Adsorbable solvent in a adsorbable solvent-insoluble particle complex means a solvent that adsorbs to the insoluble particle often resulting from polar forces that reduce or prevent the adsorbable solvent from mixing with any additional solvent in the skin care composition. The adsorbable solvent, therefore, is the solvent that has the greatest affinity (i.e., greatest adsorbability) for the insoluble particle in comparison to any other solvent in the skin care composition. Solubility parameter distance of the adsorbable solvent as it relates to the component with malodor targeted for scavenging, Ra, may be calculated from the following formula:

Ra=(4(δ_{D1}−δ_{D2})²+(δₚ₁−δₚ₂)²+(δ_{H1}−δ_{H2})²)^{1/2}

where δ_{D1} is malodor component dispersion cohesion energy, δ_{D2} is total solvent dispersion cohesion energy, δₚ₁ is malodor component polar cohesion energy, δₚ₂ is total solvent dispersion cohesion energy, δ_{H1} is malodor component hydrogen bonding cohesion energy and δ_{H2} is total solvent hydrogen bonding cohesion energy. An additional description of solubility parameter distance may be found in Hansen Solubility Parameters, Hansen, C. M., Chapter 1, CRC Press, 2000, the disclosure of which is incorporated herein by reference. Scavanged, as used herein, means attracted and/or adsorbed to so that an undesirable characteristic, like malodor, can be reduced or eliminated.

### DETAILED DESCRIPTION OF THE PREFERRED EMBODIMENTS

There is no limitation with respect to the ingredient (that can degrade to yield a component with malodor) that may be used in this invention as long the ingredient is one that can be applied to skin and/or hair and provide a benefit.

Illustrative and non-limiting examples of the type of ingredients (including active ingredients) that can degrade to yield a component with malodor and that may be used in this invention include unsaturated fatty acids, and especially, polyunsaturated fatty acids like linoleic, eicosapolyenoic, docosapolyenoic and conjugated linoleic acid (CLA). CLA can comprise a group of positional and geometric isomers of linoleic acid in which various configurations of cis and trans double bonds at positions (6,8), (7,9), (8,10), (9,11), (10,12), (11,13), or mixtures thereof are possible. Therefore, many individual isomers and combinations of isomers may be used as the active ingredient (that yields malodor) in this invention.

A preferred CLA suitable for use in the compositions made in accordance with the present invention is the cis 9, trans 11 (hereinafter referred to as c9, t11) isomer. This particular isomer of the free acid has the structure shown below:

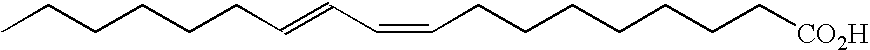

The invention also includes for use as actives derivatives of the free acid (which often comprise conjugated linoleic acid moieties) that can generate a compound with malodor. Preferable derivatives include those derived from substitution of the carboxyl group of the acid, such as esters (e.g., retinyl esters, triglyceride esters, monoglycerides esters, diglyceride esters, phosphoesters), amides (e.g., ceramide derivatives), salts (e.g., alkali metal and alkali earth metal salts, ammonium salts); and/or those derived from substitution of the C18 carbon chain, such as alpha and/or beta alkoxy and/or hydroxy derivatives.

In the case of triglyceride ester derivatives, all positional isomers of CLA substituents on the glycerol backbone are included. The triglycerides should contain at least one CLA moiety. For example, of the three esterifiable positions on the glycerol backbone, the 1 and 2 positions may be esterified with CLA and by another lipid at position 3 or as an alternative, the glycerol backbone could be esterified by CLA at the 1 and 3 positions with another lipid at position 2. Wherever the term “conjugated linoleic acid” or “CLA” is used in this specification it is to be understood that the derivatives thereof comprising CLA moieties are also included. “CLA moieties” refer to CLA fatty acyl portion(s) of a CLA derivative.

By “c9, t11 isomer enriched CLA” is meant that at least about 30% by weight of the total CLA and/or CLA moieties present in the composition is in the form of the cis 9, trans 11 isomer. Preferably, at least about 35%, and most preferably, at least 40% to about 90% by weight of the total CLA and/or CLA moieties present in the composition, is in the form of the c9, t11 isomer, including all ranges subsumed therein.

However, in one particular preferred embodiment, cis 9, trans 11 isomer and trans 10, cis 12 isomer (or any derivatives thereof) are present as the active at a weight ratio from about 40:60 to about 60:40, and preferably, at a weight ratio from about 45:55 to about 55:45, including all ratios subsumed therein CLA type products suitable for use in this invention are made available from suppliers like Stepan under the name Neobee® and Loders Croklaan under the name Clarinol™.

The CLA and/or derivatives thereof comprising CLA moieties according to the present invention may be prepared, for example, according to the method disclosed in WO 97/18320, the disclosure of which is incorporated herein by reference.

The CLA to be employed in accordance with the present invention is typically present in the skin care composition in an effective amount. Normally, the total amount of the ingredient that can degrade to yield a component with malodor is present in an amount from about 0.00001% to about 50% by weight of the composition. More preferably, the amount is from about 0.01% to about 10%, and most preferably, from about 0.1% to about 5% by weight of the composition, including all ranges subsumed therein.

Another ingredient that can degrade to yield a component with a malodor and that is suitable for use in this invention is a monoenoic fatty acid (i.e., monounsaturated fatty acid) like cis-4-decenoic, cis-9-decenoic, cis-5-lauroleic, cis-4-dodecenoic, cis-9-tetradecenoic, cis-5-teradecenoic, cis-4-tetradecenoic, cis-9-hexadecenoic, cis-6-octadecenoic, cis-9-octadecenoic, tr-9-octadecenoic, cis-11-octadecenoic, cis-9-eicosenoic, cis-11-eicosenoic, cis-11-docosenoic, cis-13-docosenoic, cis-15-tetracosenoic acid, derivatives thereof or mixtures thereof.

The preferred monoenoic fatty acid suitable for use in this invention is cis-6-octadecenoic acid (i.e., petroselinic acid) whereby the same may be used alone, in combination with other monoenoic fatty acids and/or in combination with CLA and/or in combination with other active ingredients defined herein.

If desired for use, the amount of monoenoic acid employed in the skin care composition of this invention is often from about 0.005 to about 35%, and preferably, from about 0.01 to about 25%, and most preferably, from about 0.5 to about 6% by weight, including all ranges subsumed therein.

As to component with malodor originally formulated in the skin care composition of this invention, such a component is not limited and often is one which comprises a heteroatom, and especially, nitrogen.

The only limitations with respect to the adsorbable solvent that may be used in this invention in that the same is suitable for use in a skin care composition, is not classified as a solvent with malodor (e.g., does not possess an aroma that is similar to hexanal) and has a solubility parameter distance as it relates to the component with a malodor targeted for scavaging of less than about 20.

Illustrative and non-limiting examples of the types of adsorbable solvent that may be used in this invention include those comprising dimethicone, caprylyl methicone, wickenol, triolein, isopropyl alcohol, ethanol, dipropylene glycol, mixtures thereof, or the like. Still others include propylene glycol, tripropylene glycol, ethylene glycol, diethylene glycol, triethylene glycol, polyethylene glycol, glycerin, sorbitol, any mixtures thereof or the like. In a most preferred embodiment, the adsorbable solvent is not an aldehyde, and especially, not a linear aldehyde which has 10 carbons or less. In yet another preferred embodiment, the adsorbable solvent is dipropylene glycol (DPG).

Typically, the adsorbable solvent makes up from about 0.01 to about 35%, and preferably, from about 0.05 to about 20%, and most preferably, from about 0.1 to about 5% by weight of the skin care composition, including all ranges subsumed therein.

As to the insoluble particle that may be used, the same is only limited to the extent that it can be used in a skin care composition. Illustrative examples of the types of insoluble particles that may be used in this invention include those that comprise clays such as synthetic layered silicates, smectite minerals, fumed silicas and zeolites.

Preferred synthetic layered silicates include those prepared from salts of sodium, magnesium and lithium and sold under the name Laponite® (sodium, lithium, magnesium silicate made available by Southern Clay Products, Inc.). Illustrative and non-limiting smectite minerals that may be used in this invention include pyrophyllite, talc, vermiculite, sauconite, montronite, montmorillonite hectonite, mixtures thereof and the like.

Illustrative fumed silicas that may be used include those sold under the name Aerosil (available from Degussa AG), Cab-o-sil (available from Cabot), mixtures thereof and the like.

The zeolites that may be used in this invention usually have a pore size form about 8 to about 15 angstrons in diameter and are made available by Honeywell under the name Asensa™. Other zeolites which may be used include analcite, chabazite, heulandite, natrolite, stilbite, thomosonite, and synthetic zeolites (like those made available by a gel process or a clay process where the former uses components like sodium silicate and alumina and the latter uses kaolin).

Typically, the amount of insoluble particle used in the skin care composition of the present invention is from about 0.1 to about 10, and preferably, from about 0.1 to about 8, and most preferably, from about 0.2 to about 6 weight percent, based on total weight of the skin care composition, including all ranges subsumed therein.

Water is typically the solvent (i.e., the solvent that is used in addition to the adsorbable solvent) employed in this invention wherein water will make up the balance of the skin care composition. Regarding the adsorbable solvent, the same typically has an Ra of less than about 20, and preferably, less than about 18, and most preferably, from about 1 to about 15, including all ranges subsumed therein. Such an adsorbable solvent, again, is preferably not a linear C₁ to C₁₀ aldehyde such as hexanal. Often, the amount of adsorbable solvent employed in the skin care composition of this invention is from about 0.1 to about 25%, and preferably, from about 1 to about 12%, and most preferably, from about 1 to about 5% by weight, based on total weight of the skin care composition and including all ranges subsumed therein.

Emollient materials may serve as cosmetically acceptable carriers for the skin care composition of this invention. These may be in the form of silicone oils, natural or synthetic esters and hydrocarbons. Amounts of the emollients may range anywhere from about 0.1 to about 95%, preferably between about 1 and about 50% by weight of the composition.

Silicone oils may be divided into the volatile and nonvolatile variety. The term “volatile” as used herein refers to those materials which have a measurable vapor pressure at ambient temperature. Volatile silicone oils are preferably chosen from cyclic (cyclomethicone) or linear polydimethylsiloxanes containing from 3 to 9, preferably from 4 to 5, silicon atoms.

Nonvolatile silicone oils useful as an emollient material include polyalkyl siloxanes, polyalkylaryl siloxanes and polyether siloxane copolymers The essentially nonvolatile polyalkyl siloxanes useful herein include, for example, polydimethyl siloxanes with viscosities of from about 5×10⁻⁶ to 0.1 m²/s at 25° C. Among the preferred nonvolatile emollients useful in the present compositions are the polydimethyl siloxanes having viscosities from about 1×10⁻⁵ to about 4×10⁻⁴ m²/s at 25° C.

Another class of nonvolatile silicones are emulsifying and non-emulsifying silicone elastomers. Representative of this category is Dimethicone/Vinyl Dimethicone Crosspolymer available as Dow Corning 9040, General Electric SFE 839, and Shin-Etsu KSG-18. Silicone waxes such as Silwax WS-L (Dimethicone Copolyol Laurate) may also be useful.

Among the ester emollients are:

a) Alkyl esters of saturated fatty acids having 10 to 24 carbon atoms. Examples thereof include behenyl neopentanoate, isononyl isonanonoate, isopropyl myristate and octyl stearate.

b) Ether-esters such as fatty acid esters of ethoxylated saturated fatty alcohols.

c) Polyhydric alcohol esters. Ethylene glycol mono and di-fatty acid esters, diethylene glycol mono- and di-fatty acid esters, polyethylene glycol (200-6000) mono- and di-fatty acid esters, propylene glycol mono- and di-fatty acid esters, polypropylene glycol 2000 monostearate, ethoxylated propylene glycol monostearate, glyceryl mono- and di-fatty acid esters, polyglycerol poly-fatty esters, ethoxylated glyceryl mono-stearate, 1,3-butylene glycol monostearate, 1,3-butylene glycol distearate, polyoxyethylene polyol fatty acid ester, sorbitan fatty acid esters, and polyoxyethylene sorbitan fatty acid esters are satisfactory polyhydric alcohol esters. Particularly useful are pentaerythritol, trimethylolpropane and neopentyl glycol esters of C₁-C₃₀ alcohols.

d) Wax esters such as beeswax, spermaceti wax and tribehenin wax.

e) Sugar ester of fatty acids such as sucrose polybehenate and sucrose polycottonseedate.

Natural ester emollients principally are based upon mono-, di- and tri-glycerides. Representative glycerides include sunflower seed oil, cottonseed oil, borage oil, borage seed oil, primrose oil, castor and hydrogenated castor oils, rice bran oil, soybean oil, olive oil, safflower oil, shea butter, jojoba oil and combinations thereof. Animal derived emollients are represented by lanolin oil and lanolin derivatives. Amounts of the natural esters may range from about 0.1 to about 20% by weight of the compositions.

Hydrocarbons which are suitable cosmetically acceptable carriers include petrolatum, mineral oil, C₁₁-C₁₃ isoparaffins, polybutenes, and especially isohexadecane, available commercially as Permethyl 101A from Presperse Inc.

Fatty acids having from 10 to 30 carbon atoms may also be suitable as cosmetically acceptable carriers. Illustrative of this category are pelargonic, lauric, myristic, palmitic, stearic, isostearic, oleic, linoleic, linolenic, hydroxystearic and behenic acids.

Fatty alcohols having from 10 to 30 carbon atoms are another useful category of cosmetically acceptable carrier. Illustrative of this category are stearyl alcohol, lauryl alcohol, myristyl alcohol, oleyl alcohol and cetyl alcohol.

Thickeners can be utilized as part of the cosmetically acceptable carrier of compositions according to the present invention. Typical thickeners include crosslinked acrylates (e.g. Carbopol 982®), hydrophobically-modified acrylates (e.g. Carbopol 1382®), polyacrylamides (e.g. Sepigel 305®), acryloylmethylpropane sulfonic acid/salt polymers and copolymers (e.g. Aristoflex HMB® and AVC®), cellulosic derivatives and natural gums. Among useful cellulosic derivatives are sodium carboxymethylcellulose, hydroxypropyl methocellulose, hydroxypropyl cellulose, hydroxyethyl cellulose, ethyl cellulose and hydroxymethyl cellulose. Natural gums suitable for the present invention include guar, xanthan, sclerotium, carrageenan, pectin and combinations of these gums. Inorganics may also be utilized as thickeners, particularly clays such as bentonites and hectorites, fumed silicas, talc, calcium carbonate and silicates such as magnesium aluminum silicate (Veegum®). Amounts of the thickener may range from 0.0001 to 10%, usually from 0.001 to 1%, optimally from 0.01 to 0.5% by weight of the composition.

Adjunct humectants may be employed in the present invention. These are generally polyhydric alcohol-type materials. Typical polyhydric alcohols include glycerol, propylene glycol, dipropylene glycol, polypropylene glycol, polyethylene glycol, sorbitol, hydroxypropyl sorbitol, hexylene glycol, 1,3-butylene glycol, isoprene glycol, 1,2,6-hexanetriol, ethoxylated glycerol, propoxylated glycerol and mixtures thereof. The amount of adjunct humectant may range anywhere from 0.5 to 50%, preferably between 1 and 15% by weight of the composition.

Surfactants may also be present in compositions of the present invention. Total concentration of the surfactant when present may range from about 0.1 to about 90%, preferably from about 1 to about 40%, optimally from about 1 to about 20% by weight of the composition, and being highly dependent upon the type of personal care product. The surfactant may be selected from the group consisting of anionic, nonionic, cationic and amphoteric actives. Particularly preferred nonionic surfactants are those with a C₁₀-C₂₀ fatty alcohol or acid hydrophobe condensed with from 2 to 100 moles of ethylene oxide or propylene oxide per mole of hydrophobe; C₂-C₁₀ alkyl phenols condensed with from 2 to 20 moles of alkylene oxide; mono- and di-fatty acid esters of ethylene glycol; fatty acid monoglyceride; sorbitan, mono- and di-C₈-C₂₀ fatty acids; and polyoxyethylene sorbitan as well as combinations thereof. Alkyl polyglycosides and saccharide fatty amides (e.g. methyl gluconamides) and trialkylamine oxides are also suitable nonionic surfactants.

Preferred anionic surfactants include soap, alkyl ether sulfates and sulfonates, alkyl sulfates and sulfonates, alkylbenzene sulfonates, alkyl and dialkyl sulfosuccinates, C₈-C₂₀ acyl isethionates, C₈-C₂₀ alkyl ether phosphates, C₈-C₂₀ sarcosinates, C₈-C₂₀ acyl lactylates, sulfoacetates and combinations thereof. An often most preferred anionic surfactant is sodium dodecyl sulfate (SDS). Useful amphoteric surfactants include cocoamidopropyl betaine, C₁₂-C₂₀ trialkyl betaines, sodium lauroamphoacetate, and sodium laurodiamphoacetate.

Sunscreen agents may also be included in compositions of the present invention. Particularly preferred are such materials as ethylhexyl p-methoxycinnamate, available as Parsol MCX®, Avobenzene, available as Parsol 1789® and benzophenone-3, also known as Oxybenzone. Inorganic sunscreen actives may be employed such as microfine titanium dioxide and zinc oxide. Amounts of the sunscreen agents when present may generally range from 0.1 to 30%, preferably from 2 to 20%, optimally from 4 to 10% by weight of the composition.

Certain skin care compositions of the present invention ordinarily will contain astringent actives. Examples include aluminum chloride, aluminum chlorhydrex, aluminum-zirconium chlorhydrex glycine, aluminum sulfate, zinc sulfate, zirconium and aluminum chlorohydroglycinate, zirconium hydroxychloride, zirconium and aluminum lactate, zinc phenolsulfonate and combinations thereof. Amounts of the astringents may range anywhere from about 0.5 to about 50% by weight of the composition.

Preservatives can desirably be incorporated into the skin care compositions of this invention to protect against the growth of potentially harmful microorganisms. Particularly preferred preservatives are phenoxyethanol, methyl paraben, propyl paraben, imidazolidinyl urea, dimethyloldimethylhydantoin, ethylenediaminetetraacetic acid salts (EDTA), sodium dehydroacetate, methylchloroisothiazolinone, methylisothiazolinone, iodopropynbutylcarbamate and benzyl alcohol. The preservatives should be selected having regard for the use of the composition and possible incompatibilities between the preservatives and other ingredients. Preservatives are preferably employed in amounts ranging from 0.01% to 2% by weight of the composition.

Compositions of the present invention may include vitamins. illustrative vitamins are Vitamin A (retinol), Vitamin B₂, Vitamin B₃ (niacinamide), Vitamin B₆, Vitamin C, Vitamin E, Folic Acid and Biotin. Derivatives of the vitamins may also be employed. For instance, Vitamin C derivatives include ascorbyl tetraisopalmitate, magnesium ascorbyl phosphate and ascorbyl glycoside. Derivatives of Vitamin E include tocopheryl acetate, tocopheryl palmitate and tocopheryl linoleate. DL-panthenol and derivatives may also be employed. For purposes of this invention, vitamins where present are not considered as unsaturated materials. Total amount of vitamins when present in compositions according to the present invention may range from 0.001 to 10%, preferably from 0.01% to 1%, optimally from 0.1 to 0.5% by weight of the composition.

Another type of useful substance can be that of an enzyme such as amylases, oxidases, proteases, lipases and combinations. Particularly preferred is superoxide dismutase, commercially available as Biocell SOD from the Brooks Company, USA.

Skin lightening compounds may be included in the compositions of the invention. Illustrative substances are placental extract, lactic acid, niacinamide, arbutin, kojic acid, ferulic acid, resorcinol and derivatives including 4-substituted resorcinols and combinations thereof. Amounts of these agents may range from about 0.1 to about 10%, preferably from about 0.5 to about 2% by weight of the composition.

Desquamation promoters may be present. Illustrative are the alpha-hydroxycarboxylic acids and beta-hydroxycarboxylic acids. The term “acid” is meant to include not only the free acid but also salts and C₁-C₃₀ alkyl or aryl esters thereof and lactones generated from removal of water to form cyclic or linear lactone structures. Representative acids are glycolic, lactic and malic acids. Salicylic acid is representative of the beta-hydroxycarboxylic acids. Amounts of these materials when present may range from about 0.01 to about 15% by weight of the composition.

A variety of herbal extracts may optionally be included in compositions of this invention. The extracts may either be water soluble or water-insoluble carried in a solvent which respectively is hydrophilic or hydrophobic. Water and ethanol are the preferred extract solvents. Illustrative extracts include those from green tea, chamomile, licorice, aloe vera, grape seed, citrus unshui, willow bark, sage, thyme and rosemary.

Also included may be such materials as lipoic acid, retinoxytrimethylsilane (available from Clariant Corp. under the Silcare 1M-75 trademark), dehydroepiandrosterone (DHEA) and combinations thereof. Ceramides (including Ceramide 1, Ceramide 3, Ceramide 3B and Ceramide 6) as well as pseudoceramides may also be useful. Amounts of these materials may range from about 0.000001 to about 10%, preferably from about 0.0001 to about 1% by weight of the composition.

Colorants, opacifiers and abrasives may also be included in compositions of the present invention. Each of these substances may range from about 0.05 to about 5%, preferably between 0.1 and 3% by weight of the composition.

The compositions of the present invention can also be, optionally, incorporated into an insoluble substrate for application to the skin such as in the form of a treated wipe.

A wide variety of packaging can be employed to store and deliver the skin care compositions. Packaging is often dependent upon the type of personal care end-use. For instance, leave-on skin lotions and creams, shampoos, conditioners and shower gels generally employ plastic containers with an opening at a dispensing end covered by a closure. Typical closures are screw-caps, non-aerosol pumps and flip-top hinged lids. Packaging for antiperspirants, deodorants and depilatories may involve a container with a roll-on ball on a dispensing end. Alternatively these types of skin care compositions may be delivered in a stick composition formulation in a container with propel-repel mechanism where the stick moves on a platform towards a dispensing orifice. Metallic cans pressurized by a propellant and having a spray nozzle serve as packaging for antiperspirants, shave creams and other personal care products. Toilette bars may have packaging constituted by a cellulosic or plastic wrapper or within a cardboard box or even encompassed by a shrink wrap plastic film. All of the aforementioned are considered packaging within context of the present invention.

The Examples are provided to facilitate an understanding of the present invention and they are not meant to limit the scope of the claims.

### EXAMPLE 1

Emulsions (i.e., skincare compositions) comprising CLA {50% cis 9, trans 11 and 50% trans 10, cis 12} were prepared by mixing CLA, sodium dodecyl sulfate, SDS, (about 0.6%) and water. The resulting mixture was stirred on a stirring plate for about 20 minutes in order to dissolve the SDS into solution. The SDS solution with dispersed CLA was sonicated for about 2 minutes using an Ulbra Cell™ sonicator to yield stable emulsions. A first stable emulsion was completed by adding about 2% DPG, a second was completed by adding about 2.5% clay (Laponite®) and a third was completed by adding 2% DPG and 2.5% clay (Laponite®). All completed emulsions were subjected to an additional 2 minutes of sonication. About 3% CLA was present in each emulsion, water was added to balance and all percents are percents by weight based on total weight of the emulsion.

### EXAMPLE 2

Headspace analysis was performed on the CLA emulsions prepared in Example 1. Solid phase microextration (SPME)-gas chromatography (GC) 6890 mass spectrometry (MS) 5973/flame ionization detector (FID) was used to identify the chemical composition of the vapor (i.e., aldehydes resulting from, for example, the oxidation of CLA) over aged (i.e., greater than about 3 months old and stored at about 60° C.) emulsions. One gram of each of the above-described emulsions was filled in 20 ml GC headspace sampling vials sealed with caps and septum. The GC column used was an HP-5MS column from Agilent (inner diameter 0.25 mm, length 30 m, stationary phase thickness 0.25 um). GC conditions were such that the injector was in the splitless mode with helium gas as the carrier gas. The injection port was heated to 250° C., with purge flow at split vent 50 ml/min for 2 minutes. The column was set at a constant flow mode, a 1.3 ml/min flow rate. Oven temperature ramp was held at 75° C. for 2 minutes, and increased at a rate of 6° C./min to 100° C., 1.5° C./min to 150° C., 3° C./min to 190° C., 30° C./min to 300° C. and hold for 2 minutes. MS conditions were such that solvent delay was for 0.5 minutes and scan started from low mass 35 to high mass 300. The autosampler conditions were such that samples were incubated at about 35° C. for 25 minutes with no agitation. SPME fiber was inserted into the sample headspace for a 5 minutes extraction and subsequently injected into the injector for a 15 minute desorption.

The results in the table below demonstrate that the use of insoluble particle and adsorbable solvent unexpectedly show a reduction in malodor that is greater than the sum of malodor reduction for compositions that only contain insoluble particle and only contain adsorbable solvent.

TABLE

| | Insoluble Particle | |
|---|---|---|
| Component with | and/or Adsorbable | |
| Malodor | Solvent | Malodor in Headspace |
| Butanal | Control | 1 |
| Butanal | DPG | 0.88 |
| Butanal | Laponite | 0.1 |
| Butanal | Laponite + DPG | 0.05 |
| Pentanal | Control | 1 |
| Pentanal | DPG | 0.93 |
| Pentanal | Laponite | 0.22 |
| Pentanal | Laponite + DPG | 0.05 |
| N-hexanal | Control | 1.0 |
| N-hexanal | DPG | 0.96 |
| N-hexanal | Laponite | 0.3 |
| N-hexanal | Laponite + DPG | 0.05 |
| Heptanal | Control | 1.0 |
| Heptanal | DPG | 0.94 |
| Heptanal | Laponite | 0.35 |
| Heptanal | Laponite + DPG | 0.1 |
| 2-Octenal | Control | 1 |
| 2-Octenal | DPG | 0.9 |
| 2-Octenal | Laponite | 0.3 |
| 2-Octenal | Laponite + DPG | 0.06 |
| Nonenal | Control | 1 |
| Nonenal | DPG | 0.9 |
| Nonenal | Laponite | 0.22 |
| Nonenal | Laponite + DPG | 0.05 |

What is claimed is:

1. A method for reducing malodor in a skin care composition comprising the steps of:
(a) formulating the skin care composition with a component with a malodor or an ingredient that can degrade to yield a component with a malodor; and
(b) including in the skin care composition an insoluble particle and an adsorbable solvent, the component with a malodor being one suitable to hydrogen bond with the insoluble particle and/or be scavanged by the adsorbable solvent in an adsorbable solvent-insoluble particle complex
wherein the insoluble particle has a surface area from about 75 to about 3500 m²/g and the adsorbable solvent has a solubility parameter distance from the component with a malodor, Ra, of less than about 20.

2. The method according to claim 1 wherein the component with a malodor comprises a heteroatom.

3. The method according to claim 1 wherein the component with a malodor comprises nitrogen.

4. The method according to claim 1 wherein the ingredient that can degrade to yield a component with malodor is a polyunsaturated fatty acid, a monounsaturated fatty acid, or a mixture thereof.

5. The method according to claim 4 wherein the polyunsaturated fatty acid comprises linoleic acid, conjugated linoleic acid, eicosapolyenoic acid, docosapolyenoic acid or a mixture thereof.

6. The method according to claim 4 wherein the polyunsaturated fatty acid comprises conjugated linoleic acid.

7. The method according to claim 4 wherein the monounsaturated acid comprises cis-4-decenoic, cis-9-decenoic, cis-5-lauroleic, cis-4-dodecenoic, cis-9-tetradecenoic, cis-5-teradecenoic, cis-4-tetradecenoic, cis-9-hexadecenoic, cis-6-octadecenoic, cis-9-octadecenoic, tr-9-octadecenoic, cis-11-octadecenoic, cis-9-eicosenoic, cis-11-eicosenoic, cis-11-docosenoic, cis-13-docosenoic, cis-15-tetracosenoic acid, derivatives thereof or mixtures thereof.

8. The method according to claim 4 wherein the monounsaturated fatty acid is petroselinic acid.

9. The method according to claim 1 wherein the insoluble particle comprises clay, smectite, silica, zeolite or mixtures thereof.

10. The method according to claim 1 wherein the adsorbable solvent comprises dimethicone, caprylyl methicone, wickenol, triolein, isopropyl alcohol, ethanol, dipropylene glycol, mixtures thereof, or the like. Still others include propylene glycol, tripropylene glycol, ethylene glycol, diethylene glycol, triethylene glycol, polyethylene glycol, glycerin, sorbitol, or mixtures thereof.

11. The method according to claim 1 wherein the adsorbable solvent is dipropylene glycol.

12. The method according to claim 1 wherein the skin care composition comprises from about 0.01 to about 35% by weight adsorbable solvent and from about 0.1 to about 10% by weight insoluble particle.

13. A skin care composition comprising:
(a) carrier;
(b) insoluble particle;
(c) adsorbable solvent; and
(d) a component with a malodor,
wherein the component with a malodor is one which is suitable to hydrogen bond with the insoluble particle and/or be scavanged by the adsorbable solvent in an adsorbable solvent-insoluble particle complex further wherein the insoluble particle has a surface area from about 75 to about 3500 m²/g and the adsorbable solvent has a solubility parameter distance from the component with a malodor, Ra, of less than about 20.

14. The skin care composition according to claim 1 wherein the insoluble particle comprises clay, smectite, silica, zeolite or a mixture thereof.

15. The skin care composition according to claim 1 wherein the adsorbable solvent comprises dimethicone, caprylyl methicone, wickenol, triolein, isopropyl alcohol, ethanol, dipropylene glycol, mixtures thereof, or the like. Still others include propylene glycol, tripropylene glycol, ethylene glycol, diethylene glycol, triethylene glycol, polyethylene glycol, glycerin, sorbitol, any mixtures thereof or the like.

16. The skin care composition according to claim 13 wherein the component with a malodor comprises a heteroatom.

17. The skin care composition according to claim 13 wherein the component with a malodor is a degradation product of a polyunsaturated acid or a monounsaturated acid or both.

18. The skin care composition according to claim 17 wherein the polyunsaturated acid comprises linoleic acid, conjugated linoleic acid, eicosapolyenoic acid, docosapolyenoic acid or mixtures thereof.

19. The skin care composition according to claim 17 wherein the monounsaturated acid comprises cis-4-decenoic, cis-9-decenoic, cis-5-lauroleic, cis-4-dodecenoic, cis-9-tetradecenoic, cis-5-teradecenoic, cis-4-tetradecenoic, cis-9-hexadecenoic, cis-6-octadecenoic, cis-9-octadecenoic, tr-9-octadecenoic, cis-11-octadecenoic, cis-9-eicosenoic, cis-11-eicosenoic, cis-11-docosenoic, cis-13-docosenoic, cis-15-tetracosenoic acid or mixtures thereof.
